(19) United States
(12) Patent Application Publication
(10) Pub. No.: US 2021/0346245 A1
(43) Pub. Date: Nov. 11, 2021

(54) SOLID COSMETIC COMPOSITION AND MANUFACTURING METHOD THEREFOR

(71) Applicant: MASSCON CO., LTD., Seoul (KR)

(72) Inventors: Yongeui LEE, Gyeonggi-do (KR); Min Young CHEONG, Gyeonggi-do (KR); Chang Bin LEE, Busan (KR)

(21) Appl. No.: 17/269,674

(22) PCT Filed: Jul. 23, 2019
(86) PCT No.: PCT/KR2019/009061

§ 371 Date: Feb. 19, 2021

(30) Foreign Application Priority Data
Aug. 23, 2018 (KR) 10-2018-0098430

(51) Int. Cl. A61K 8/02; A61K 8/92; A61K 8/65; A61K 8/25; A61K 8/73; A61Q 19/00

(52) U.S. Cl. CPC A61K 8/0216; A61K 8/0279; A61K 8/922; A61K 8/65; A61K 8/25; A61K 8/731; A61K 2800/412; A61K 8/735; A61Q 19/00; A61K 2800/33; A61K 2800/591; A61K 2800/60; A61K 2800/805; A61K 8/732

(57) ABSTRACT

The present invention relates to a solid cosmetic composition and a manufacturing method therefor, wherein the solid cosmetic composition comprises a high content of an active ingredient without the use of a preservative or a surfactant and can improve the processability of products. In addition, the convenience of use and portability of the solid cosmetic composition can be improved since the active ingredient can be selectively or simultaneously loaded irrespective of hydrophilicity and hydrophobicity thereof and can be dissolved or released in water without a surfactant.

### TECHNICAL FIELD

The present invention relates to a solid cosmetic composition which comprises a high content of an active ingredient without the use of a preservative or a surfactant and can improve convenience of use and portability of products.

In addition, it relates to a solid cosmetic composition and a manufacturing method capable of selectively or simultaneously producing a high content of an active ingredient irrespective of the hydrophilic (water-soluble) and hydrophobic (oil-soluble) properties thereof, and in particular, capable of improving the load rate of a hydrophobic active ingredient.

### BACKGROUND ART

Cosmetics are generally commercialized in the form of a mixture of various ingredients. As the ingredients contained are easy to be deteriorated, additives such as preservatives or antiseptics are used to secure the shelf life. Preservatives can prevent the proliferation of microorganisms in the mixture to prevent denaturation or deterioration of the efficacy of the active ingredient. However, when a cosmetic product containing such an additive is applied to the skin, it may irritate the skin. Conventional chemical preservatives include formaldehyde, methylisothiazolinone, benzophenone, paraben, and phenoxyethanol, and some of the preservatives may cause problems such as skin troubles and abnormal hormone secretion when used for a long time.

In addition, since cosmetics are commercialized in which an oil-soluble active ingredient and a water-soluble active ingredient are mixed each other, a surfactant is essentially required to uniformly mix the active ingredients. As surfactants reduce the important skin barrier function of the epidermis while washing off epidermal lipids with a strong cleansing power, moisture in the skin evaporates quickly, resulting in dry skin and skin aging. When the skin barrier becomes impaired, it can also cause skin problems. In addition, the surfactants are known to have various harmful effects such as promoting skin aging by infiltrating various ingredients or drugs such as chemical additives, fragrances, and pigments contained in cosmetics into the skin. In order to improve this, many studies have been conducted on natural surfactants, but there is still a need for improvement in emulsifying power and cleansing power.

In order to solve this problem, conventionally, a method of capturing or encapsulating an active ingredient, for example, in a liposome structure has been used. However, this method has disadvantages that dispersing in water is required, the load rate of poorly soluble substances is limited, and a carrier optimized for each characteristic according to hydrophilicity or hydrophobicity of the loaded active ingredient is required. Therefore, the use of the minimum required amounts of surfactants is inevitable.

Many studies have been attempted to gel or solidify cosmetic compositions for convenience in use and portability without use of preservatives or surfactants described above. However, these methods are disadvantages in that it is generally difficult to manufacture cosmetics containing high content of an active ingredient, gelling agents and additives are required or a certain process should be undergone, and products have the lowered feeling of use and efficacy due to the low content of active ingredients.

Therefore, there is a need for developing a cosmetic composition which can improve the efficacy of active ingredients while minimizing the use of additives, freely shape the formulations to improve portability and convenience of use, selectively or simultaneously produce a cosmetic containing a high content of an active ingredient irrespective of the hydrophilic and hydrophobic properties thereof, and in particular, improve the load rate of a hydrophobic active ingredient.

### DETAILED DESCRIPTION OF THE INVENTION

### Technical Problem

It is an object of the present invention to provide a solid cosmetic composition and a manufacturing method capable of improving efficacy of an active ingredient by including a high content of hydrophilic and hydrophobic active ingredients without a preservative or a surfactant and improving portability and convenience of use.

### Solution to Problem

In order to solve the above problem, the present invention is to provide a solid cosmetic composition comprising:

porous particles having a plurality of pores;

a binder for binding the porous particles to each other; and

a cosmetic active ingredient contained in the pores of the porous particles,

wherein a weight ratio of the binder to the porous particles is 1:5 to 95.

According to one embodiment, the porous particles may be at least one selected from the group consisting of silica, zeolite, activated carbon and acrylic resin particles.

According to one embodiment, the binder may be at least one selected from the group consisting of agar, carrageenan, gellan gum, gelatin, collagen, hyaluronic acid, lactose, microcrystalline cellulose, starch, calcium phosphate, soap, and wax.

According to one embodiment, an average particle size of the porous particle may be 1 to 100 μm.

According to one embodiment, an average pore diameter of the porous particle is 1 to 100 nm.

According to one embodiment, the cosmetic active ingredient may be oil-soluble and the load rate of the oil-soluble active ingredient may be 100 to 500% as calculated according to Equation 1.

Load rate (%)=(a maximum weight of the loaded oil-soluble active ingredient(g)/a weight of porous particles(g))×100 [Equation 1]

According to one embodiment, the structure formed by the porous particles and the binder may have a void and an average diameter of the void may be 0.1 to 100 μm.

In addition, a water-soluble cosmetic active ingredient may be contained in the void.

Another aspect of the present invention, there is provided a method for manufacturing a solid cosmetic composition, which is a wet manufacturing process, the method comprising:

1) dispersing porous particles in water;

2) adding a water-soluble active ingredient and a binder to the dispersion of 1) and homogeneously mixing them at a high temperature;

3) injecting the mixture of 2) into a casting and molding it by cooling;

4) drying for removal of water; and

5) loading an oil-soluble active ingredient into the mixture of 4) in which water is removed,

wherein a weight ratio of the binder to the porous particles is 1:5 to 95.

Another aspect of the present invention, there is provided a method for manufacturing a solid cosmetic composition, which is a dry manufacturing process, the method comprising:

1) homogeneously mixing porous particles, a powder-type water-soluble active ingredient and a binder;

2) tableting the mixture of 1) to prepare a molded product; and

3) loading an oil-soluble active ingredient into the molded product of 2),

wherein a weight ratio of the binder to the porous particles is 1:5 to 95.

Other specifics of the embodiments of the present invention are included in the detailed description below.

### Effect of the Invention

According to the solid cosmetic composition and its manufacturing method of the present invention, it is possible to manufacture a solid cosmetic product containing a high content of an active ingredient without the use of preservatives or surfactants, thereby improving efficacy of water-soluble and oil-soluble active ingredients and improving convenience in use and portability.

### BRIEF DESCRIPTION OF DRAWINGS

FIG. 1 schematically shows a structure of the composition according to the present invention.

FIG. 2 is a photograph showing the appearance of the composition according to Example 1.

FIG. 3 is a scanning electron microscope (SEM) photograph of silica and the composition according to Example 1.

FIG. 4 is a microscope photograph under transmission mode and reflection mode of the composition according to Example 1.

### BEST MODE FOR CARRYING OUT THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives frilling within the spirit and scope of the invention. In the following description of the present invention, detailed description of known functions will be omitted if it is determined that it may obscure the gist of the present invention.

Among most active ingredients used in cosmetics, an oil component is dispersed in water or filled in capsules to maximize its effect. In this case, surfactants and preservatives are essentially added. These surfactants and preservatives may cause skin troubles or cause environmental pollution even after washing. In addition, cosmetic products in a liquid or cream form undergo deterioration and contamination of active ingredients as they are exposed to the external environment in the process of repeatedly opening and taking out.

In order to solve this problem, the present invention is intended to overcome the limitations of cosmetic products in a liquid or cream form. Therefore, the present invention provides a solid cosmetic composition that can be used for single use with minimizing or eliminating additives such as preservatives and surfactants, thereby preventing deterioration of active ingredients contained in cosmetics to maintain freshness and improve portability. In addition, since the existing solid cosmetics have a limitation in that the content of an active ingredient is low, it is intended to improve the efficacy of the active ingredient by manufacturing solid cosmetics containing a high content of the active ingredient.

Hereinafter, the solid cosmetic composition and the method of manufacturing the same according to embodiments of the present invention will be described in more detail.

As used herein, the term “binding agent” may be described interchangeably with “matrix”, “anchor” or “binder” and may refer to any material having a function of maintaining a shape of the product, for example any material having a fibrous structure, which can exist in inter-particle spaces to form a void.

Unless otherwise specified in the disclosure, the expression “to” as used with numerical values means an expression including the corresponding numerical value. Specifically, for example, the expression “1 to 2” means not only including 1 and 2, but also including all numerical values between 1 and 2.

The present invention provides a solid cosmetic composition, comprising porous particles having a plurality of pores, a binder for binding the porous particles to each other, and a cosmetic active ingredient contained in the pores of the porous particles.

The weight ratio of the porous particles and the binder may affect the load rate of the active ingredient and molding stability. Specifically, when the weight ratio of the binder is excessively increased, it may be difficult to release or dissolve in a solvent such as water when used due to binding with a gel-like structure in the solid cosmetic composition. Conversely, when the weight ratio of the binder is excessively reduced, it may be difficult to hold the shape. That is, when the weight ratio of the binder is excessively increased, the amount of porous particle to load the active ingredient in the solid cosmetic composition decreases, so that the load rate may be lowered. Conversely, when the weight ratio of the binder is excessively reduced, it may be difficult to hold the shape of ‘pill’ or ‘tablet’. Therefore, according to one embodiment, the weight ratio of the binder to the porous particles is 1:5 to 95, for example 1:10 to 50, for example 1:20 to 50, for example 1:30 to 50.

According to one embodiment, the porous particles may comprise at least one selected from the group consisting of silica, zeolite, activated carbon, and acrylic resin particles. For example, the acrylic resin particles may include poly(methylmethacrylate)(PMMA).

As the porous particle, it is preferable to use a material that is harmless to human body and environment. An additional carrier can be further included. According to one embodiment, the additional carrier may include amphiphilic polymers, such as amphiphilic copolymers having both hydrophobic polyester blocks and hydrophilic blocks, which are self-assemble. Specifically, the additional carrier including amphiphilic polymers can allow polymers having hydrophilic molecules and hydrophobic molecules bonded to each other to cause microphase separation in an aqueous solution spontaneously to have nano-sized regularity. In addition, the additional carrier may include at least one selected from the group consisting of poloxamer, poly(lactide-co-glycolide) (PLGA), poloxalene, poly(oxyethylene)-poly(lactic acid), poly(oxyethylene)-polycaprolactone, methoxy poly(oxyethylene)-polycaprolactone, poly(oxyethylene)-poly(oxypropylene), poly(acrylic acid)-polystyrene, Poly(oxyethylene)-polystyrene and poly(acrylic acid)-poly(butadiene), which have hydrophilic molecules and hydrophobic molecules bonded in the form of a copolymer, for example. The additional carrier may be added in a minimum amount within a range that does not show a harmful effect to the cosmetic composition.

The porous particles have a very large surface area due to the formation of pores and form a three-dimensional skeletal structure, so that the cosmetic active ingredient can be prevented from degeneration due to exposure to the external environment such as light, air, and heat to be loaded stably. In addition, the porous particles can serve as a main component for loading the active ingredient. As such, an average pore diameter of the particles for effective loading and releasing may be, for example, 1 to 100 nm, for example, 10 to 100 nm, for example, 20 to 50 nm, and the average particle size (diameter) may be 1 to 100 μm, for example, 10 to 100 μm. In addition, in order to improve the load rate, the porosity of the porous particles may be, for example, 70% or more, for example, 80% or more, for example, 90% or more. The load rate on the porous particles may be calculated according to Equation 1, and may have, for example, 100 to 500%, for example, 200 to 500%.

Load rate (%)=(a maximum weight of the loaded oil-soluble active ingredient(g)/a weight of porous particles(g))×100 [Equation 1]

According to one embodiment, the binder may comprise at least one selected from the group consisting of agar, carrageenan, gellan gum, gelatin, collagen, lactose, microcrystalline cellulose, starch, calcium phosphate, soap, and wax. For example, in the case of collagen, it may include fibrous collagen because it should be able to hold a gel matrix structure.

The binder may have, for example, a fibrous structure, and may be a material having both a function of an active ingredient and hydrophilic properties, such as collagen, hyaluronic acid, etc. In addition, the binder plays a role of stably binding porous particles to each other and is characterized by a lot of voids formed between structures entangled with particles. The average void diameter of the structure thus formed may vary depending on the binder used, and it may be, for example, 0.1 to 100 μm, for example, 0.5 to 50 μm, for example, 1 to 20 μm. With such a void size, it can serve as a channel for fixing porous particles and for loading and releasing the active ingredient. Moreover, incorporation of the binder can hold the shape of the product. Depending on the binder used, the shape of the product can be loosened immediately by mixing with a small amount of water for one use or can be maintained when used several times.

According to one embodiment, the cosmetic active ingredient refers to an ingredient that exhibits beneficial effects on the skin and may include a substance that can play a role such as whitening, moisturizing, anti-inflammatory, anti-aging, and UV protection, for example vegetable oils, animal oils, extracts, etc. as an oil-soluble active ingredient. The vegetable oil may include oils obtained from, for example, coconut, jojoba, olive, almond, avocado, macadamia, evening primrose, rosehip, apricot seed, sunflower, camellia, etc., but it is not limited as long as it is a vegetable oil that can be generally included in cosmetics.

In addition, the animal oil may include, for example, mink oil, emu oil, horse oil, etc., and it is not limited as long as it is an animal oil that can be generally included in cosmetics.

In addition, the oil-soluble or water-soluble active ingredient to be included may be exemplified by water-soluble collagen, hyaluronic acid, aloe, squalan, ceramide, vitamins, vitamin precursors, polysaccharides, minerals, arbutin, ethyl ascorbyl ether, ascorbyl glucoside, ascorbyl tetraisopalmitate, magnesium ascorbyl phosphate, niacinamide, alpha-bisabolol, retinol, retinyl palmitate, polyethoxylated retinamide, adenosine, etc. Conventional adjuvants such as moisturizers, antioxidants, pigments, fragrances, etc. may be further included.

These cosmetic active ingredients may have hydrophobic or hydrophilic properties. The composition according to the present invention may selectively support a hydrophobic active ingredient or a hydrophilic active ingredient, and simultaneously support a hydrophobic active ingredient and a hydrophilic active ingredient.

As a specific example, a hydrophobic or oil-soluble active ingredient may be supported in the porous particles, and a hydrophilic active ingredient may be supported in the space (void) between the composite structures in which the porous particles and the binder are bonded and arranged. This is according to some embodiments, and the present invention is not limited to the above.

Another aspect of the present invention, there is provided a method for manufacturing a solid cosmetic composition, which is a wet manufacturing process, the method comprising:

1) dispersing porous particles in water;

2) adding a water-soluble active ingredient and a binder to the dispersion of 1) and homogeneously mixing them at a high temperature;

3) injecting the mixture of 2) into a casting and molding it by cooling;

4) drying for removal of water; and

5) loading an oil-soluble active ingredient into the mixture of 4) in which water is removed,

wherein a weight ratio of the binder to the porous particles is 1:5 to 95.

According to one embodiment, the weight ratio of the porous particles to binder in the wet manufacturing process may be, for example, 5 to 95, for example 10 to 50, for example 20 to 90.

According to one embodiment, the step of homogeneous mixing at a high temperature may be performed at, for example, 50 to 200° C., for example 70 to 150° C., for example 80 to 110° C., for example 50 to 60° C. In the temperature range as described above, the structures of the porous particles and the binder may be more homogenized and stably formed.

Another aspect of the present invention, there is provided a method for manufacturing a solid cosmetic composition, which is a dry manufacturing process, the method comprising:

1) homogeneously mixing porous particles, a powder-type water-soluble active ingredient and a binder;

2) tableting the mixture of 1) to prepare a molded product; and

3) loading an oil-soluble active ingredient into the molded product of 2),

wherein a weight ratio of the binder to the porous particles is 1:5 to 95.

According to one embodiment, the weight ratio of the binder and the porous particles in the dry manufacturing process may be, for example, 1:5 to 95, for example 1:20 to 90.

The cosmetic composition of the present invention may be prepared in any formulation commonly used in the art, for example, may have various types of preparations such as cleansers, peeling agents, packs, etc., and for example, it can be processed into a shape such as a gel, balm, stick, tube, tablet, pill, etc. In addition, when used, it can be converted into a gel, foam, or liquid by moisture, and for example, it can be used with a small amount of water to release and deliver the active ingredient to the skin. However, this is according to some embodiments, and the present invention is not limited to the above.

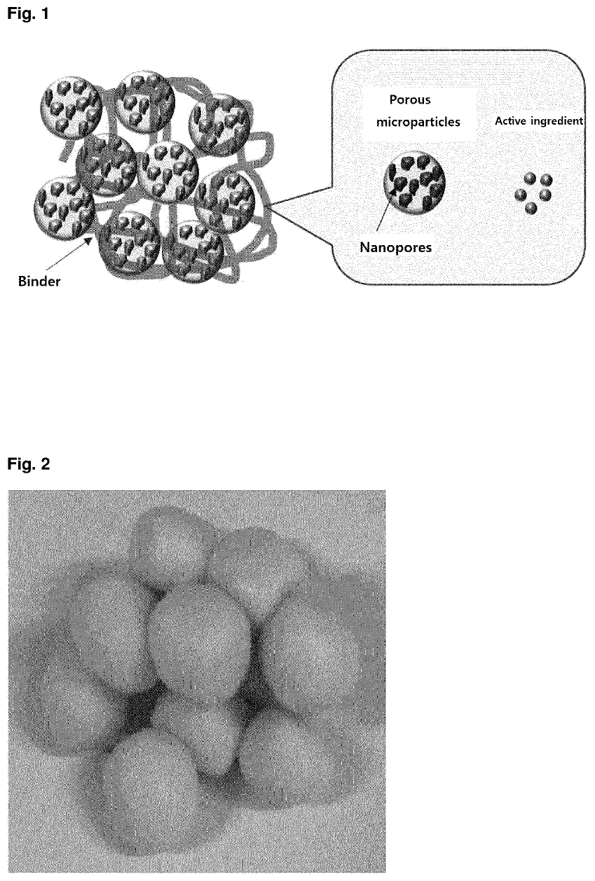

The solid cosmetic composition of the present invention manufactured according to the above method may have a structure in which porous particles are distributed in the binder, as shown in FIG. 1. In addition, by loading cosmetic active ingredients in the pores of the porous particles and the voids of the binder structures, it is possible to load a high content of the active ingredient, as well as to prevent exposure to external environments such as light, air, heat, etc. By subdividing and processing the amount of single use, it is possible to improve convenience of use while maintaining freshness.

Hereinafter, embodiments of the present invention will be described in detail so that those skilled in the art can easily carry out the present invention. The present invention may, however, be embodied in many different forms and should not be construed as limited to the embodiments set forth herein.

### Examples 1 to 4

A solid cosmetic composition was prepared according to the composition shown in Table 1.

TABLE 1

| | Cosmetic | Cosmetic | | | Weight | |
|---|---|---|---|---|---|---|
| | active | active | | | ratio of | |
| | ingredient | ingredient | | | binder:Porous | Manufacturing |
| Ex. | (hydrophobic) | (hydrophilic) | Porous particle | Binder | particles | process |
| Example 1 | Jojoba | | Silica 5 g | Agar 0.5 g | 1:10 | Wet |
| | oil | | (Average pore | | | |
| | | | diameter: 30~40 nm) | | | |
| Example 2 | Jojoba | | Silica 5 g | Agar 0.25 g | 1:20 | Wet |
| | oil | | (Average pore | | | |
| | | | diameter: 30~40 nm) | | | |
| Example 3 | Jojoba | | Silica 5 g | Agar 0.1 g | 1:50 | Wet |
| | oil | | (Average pore | | | |
| | | | diameter: 30~40 nm) | | | |
| Example 4 | Jojoba | Water- | Silica 5 g | Agar 0.25 g | 1:20 | Wet |
| | oil | soluble | (Average pore | | | |
| | Lavender | collagen | diameter: 30~40 nm) | | | |
| | oil | | | | | |
| Example 5 | Jojoba | | Silica 5 g | Microcrystalline | 1:10 | Dry |
| | oil | | (Average pore | cellulose 0.5 g | | |
| | | | diameter: 30~40 nm) | | | |
| Example 6 | Jojoba | Water- | Silica 5 g | Microcrystalline | 1:10 | Dry |
| | oil | soluble | (Average pore | cellulose 0.5 g | | |
| | | collagen | diameter: 30~40 nm) | | | |
| Comp. | Jojoba | | Silica 5 g | Agar 2g | 1:2.5 | Wet |
| Example 1 | oil | | (Average pore | | | |
| | | | diameter: 30~40 nm) | | | |
| Comp. | Jojoba | | Silica 5 g | Agar 0.05 g | 1:100 | Wet |
| Example 2 | oil | | (Average pore | | | |
| | | | diameter: 30~40 nm) | | | |
| Comp. | Jojoba | | Silica 5 g | Microcrystalline | 1:2.5 | Dry |
| Example 3 | oil | | (Average pore | cellulose 2 g | | |
| | | | diameter: 30~40 nm) | | | |
| Comp. | Jojoba | | Silica 5 g | Microcrystalline | 1:100 | Dry |
| Example 4 | oil | | (Average pore | cellulose 0.05 g | | |
| | | | diameter: 30~40 nm) | | | |

Porous silica (average particle size: 5-15 μm) was used as porous particles and extracts of fish scales (molecular weight 500 Da or less) were used as a water-soluble collagen.

### Example 1

The porous particles were placed in a 70 ml vial and agar and 30 g of purified water were added thereto, followed by stirring. After raising the temperature of the vial to 100° C. and stirring for 30 minutes, it was poured into a mold and left at room temperature for preferably at least 1 hour, for example for 2 hours. The molded mixture was dried in a convection oven at 70° C. for 5 hours or more to completely remove moisture, and then jojoba oil was added to the prepared carrier and allowed to stand until the oil was sufficiently absorbed. The load rate of the active ingredient was measured based on the maximum amount of jojoba oil that can be absorbed while increasing the amount of jojoba oil. A visual observation photograph of the prepared composition is shown in FIG. 2. Examples 2 to 6 showed similar appearance to that of Example 1.

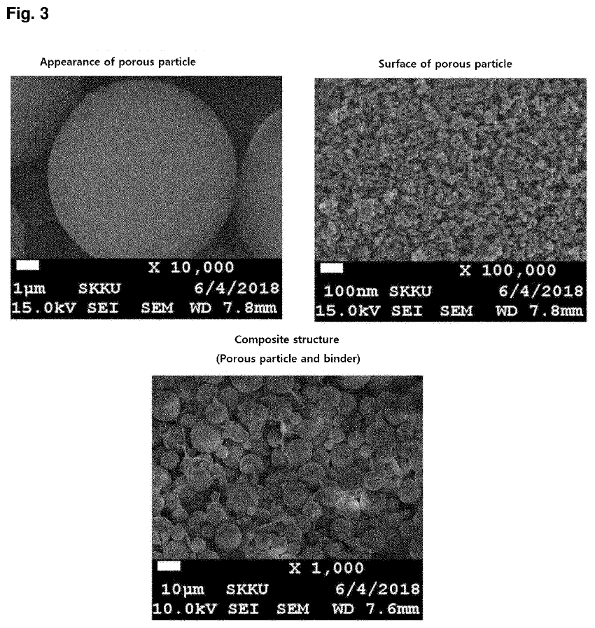

In addition, a scanning electron microscope (SEM) photograph of the silica used in Examples and the composition according to Example 1 is shown in FIG. 3. As shown in FIG. 3, it can be seen that the pore diameter of silica is 30 to 40 nm, and the diameter of voids between the silica particles are approximately 10 μm.

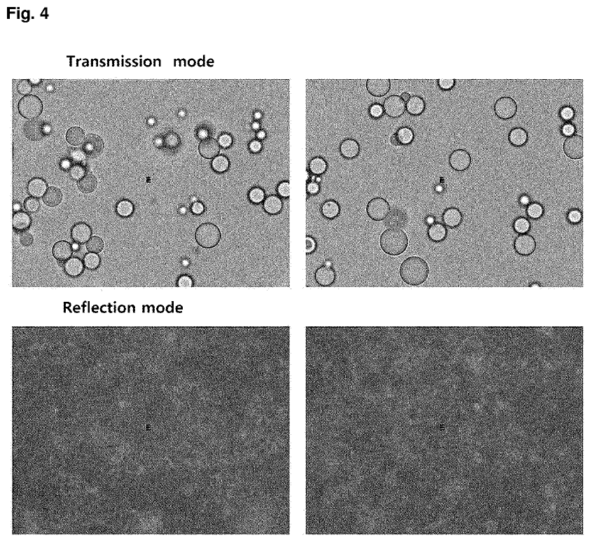

In addition, transmission mode and reflection mode observation photos with a microscope (BX53M/Olympus) for Example 1 are shown in FIG. 4, respectively. In the transmission mode of FIG. 4, the black line of the outline represents the outline of the particle, and the inside thereof represents pores, and in the reflection mode, the white line of the outline represents the outline of the particle, and the inside black part represents pores.

### Example 2

A composition was prepared in the same manner as in Example 1, except that the porous particles were placed in a 70 ml vial, and 0.25 g of agar and purified water were added and then stirred.

### Example 3

A composition was prepared in the same manner as in Example 1, except that the porous particles were placed in a 70 ml vial, and 0.1 g of agar and purified water were added and then stirred.

### Example 4

A composition was prepared in the same manner as in Example 1, except that the porous particles were placed in a 70 ml vial, and agar, 5 g of a water-soluble collagen and 50 g of purified water were added and then stirred.

In addition, the composition was prepared in the same manner as in Example 1, except that jojoba oil was added with maintaining the weight ratio of lavender oil to jojoba oil of 1:1.

### Example 5

Porous particles were placed in a 70 ml vial, and microcrystalline cellulose (average particle size of 100 μm, density 0.3-0.5 g/cm³) was added and stirred uniformly. This mixture was prepared into a tablet of a predetermined size using a manual tablet press. Jojoba oil was added to the tablet-shaped carrier and allowed to stand until the oil was sufficiently absorbed. The load rate of the active ingredient was measured based on the maximum amount of jojoba oil that can be absorbed while increasing the amount of jojoba oil.

### Example 6

A composition was prepared in the same manner as in Example 5, except that the porous particles were placed in a 70 ml vial, and additionally 5 g of a water-soluble collagen was added and then stirred.

### Comparative Example 1

A composition was prepared in the same manner as in Example 1, except that the porous particles were placed in a 70 ml vial, and 2 g of agar and purified water were added and then stirred.

### Comparative Example 2

A composition was prepared in the same manner as in Example 1, except that the porous particles were placed in a 70 ml vial, and 0.05 g of agar and purified water were added and then stirred.

### Comparative Example 3

A composition was prepared in the same manner as in Example 5, except that the porous particles were placed in a 70 ml vial, and 2 g of microcrystalline cellulose was added and then stirred.

### Comparative Example 4

A composition was prepared in the same manner as in Example 5, except that the porous particles were placed in a 70 ml vial, and 0.05 g of microcrystalline cellulose was added and then stirred.

### Experimental Example 1: Measurement of load rate of hydrophobic (oil-soluble) active ingredient

In order to measure the load rate of the active ingredient for compositions from Examples and Comparative Examples, the amount of the loaded active ingredient relative to the weight of the porous particles was measured.

The load rate was calculated according to Equation 1, and the results are shown in Table 2.

Load rate (%)=(a maximum weight of the loaded active ingredient(g)/a weight of porous particles(g))×100 [Equation 1]

TABLE 2

| Ex. | Load rate of active ingredient (%) |
|---|---|
| Comparative Example 1 | 150% |
| Comparative Example 2 | — |
| Comparative Example 3 | 120% |
| Comparative Example 4 | — |
| Example 1 | 200% |
| Example 2 | 220% |
| Example 3 | 250% |
| Example 4 | 230% |
| Example 5 | 180% |
| Example 6 | 200% |

As shown in the results of the Experimental Example, it can be seen that the load rate of the hydrophobic active ingredient in Examples was significantly improved compared to Comparative Examples. When the content ratio of the porous particles that mainly load the hydrophobic (oil-soluble) active ingredient is over a certain range, the load rate increases. When the content ratio of the binder is over a certain range, not only the content ratio of the porous particles is lowered, but also the voids formed by the binder and the porous particles also become small, so that the load rate of the hydrophobic active ingredient is lowered. However, as can be seen in Comparative Examples 2 and 4, when the content ratio of the binder is too low relative to the porous particles, the molded article is unstable, so it is difficult to form a stable molded article, making it impossible to accurately measure the load rate and the stability in use is also reduced.

### Experimental Example 2: Shape Control and Stability

In order to evaluate shaping control and shape stability for each composition, each composition taken was transferred to a 70 mol vial. The sample was placed in a convention oven at 50° C. for 1 day, and the shape change was evaluated. The results are shown as OK (good) and NG (not good) in Table 3.

TABLE 3

| Ex. | Shape control | Shape stability |
|---|---|---|
| Comparative Example 1 | Moldable | OK |
| Comparative Example 2 | Not moldable | NG |
| Comparative Example 3 | Moldable | OK |
| Comparative Example 4 | Not moldable | NG |
| Example 1 | Moldable | OK |
| Example 2 | Moldable | OK |
| Example 3 | Moldable | OK |
| Example 4 | Moldable | OK |
| Example 5 | Moldable | OK |
| Example 6 | Moldable | OK |

As shown in the results of the Experimental Example, it was found that, as in Comparative Examples 2 and 4, when the content ratio of the binder to the porous particles is too low, not only molding is impossible, but also stability is lowered. In addition, it was found that, as in Comparative Examples 1 and 3, when the content ratio of the binder to the porous particles is too high, the shape control and stability are shown to be good, but the load rate of the active ingredient was considerably reduced as shown in Experimental Example 1.

As can be seen from the results as described above, the composition according to the present invention can control the shape freely while improving the load rate of active ingredients and provide excellent stability. In addition, as the shape can be freely processed, it is possible to subdivide the amount of single use, thereby improving convenience of use and portability of products. Therefore, it is possible to provide products that can meet the needs of consumers while maximizing the purpose of the product.

The above description is merely illustrative of the technical idea of the present invention, and those of ordinary skill in the art to which the present invention pertains can make various modifications and variations without departing from the essential characteristics of the present invention. In addition, the embodiments disclosed in the present invention are not intended to limit the technical idea of the present invention, but to explain the technical idea, and the scope of the technical idea of the present invention is not limited by these embodiments. The scope of protection of the present invention should be interpreted by the following claims, and all technical ideas within the scope equivalent thereto should be construed as being included in the scope of the present invention.

1. A solid cosmetic composition comprising:
porous particles having a plurality of pores;
a binder for binding the porous particles to each other; and
a cosmetic active ingredient contained in the pores of the porous particles,
wherein a weight ratio of the binder to the porous particles is 1:5 to 95.

2. The solid cosmetic composition according to claim 1, wherein the porous particles are at least one selected from the group consisting of silica, zeolite, activated carbon and acrylic resin particles.

3. The solid cosmetic composition according to claim 1, wherein the binder is at least one selected from the group consisting of agar, carrageenan, gellan gum, gelatin, collagen, hyaluronic acid, lactose, microcrystalline cellulose, starch, calcium phosphate, soap, and wax.

4. The solid cosmetic composition according to claim 1, wherein an average particle size of the porous particle is 1 to 100 μm.

5. The solid cosmetic composition according to claim 1, wherein an average pore diameter of the porous particle is 1 to 100 nm.

6. The solid cosmetic composition according to claim 1, wherein the cosmetic active ingredient is oil-soluble and the load rate of the oil-soluble active ingredient is 100 to 500% as calculated according to Equation 1.
Load rate (%)=(a maximum weight of the loaded oil-soluble active ingredient(g)/a weight of porous particles(g))×100 [Equation 1]

7. The solid cosmetic composition according to claim 1, wherein a structure formed by the porous particles and the binder has a void and an average diameter of the void is 0.1 to 100 μm.

8. The solid cosmetic composition according to claim 7, wherein a water-soluble cosmetic active ingredient is contained in the void.

9. A method for manufacturing a solid cosmetic composition, which is a wet manufacturing process, the method comprising:
1) dispersing porous particles in water;
2) adding a water-soluble active ingredient and a binder to the dispersion of 1) and homogeneously mixing them at a high temperature;
3) injecting the mixture of 2) into a casting and molding it by cooling;
4) drying for removal of water; and
5) loading an oil-soluble active ingredient into the mixture of 4) in which water is removed,
wherein a weight ratio of the binder to the porous particles is 1:5 to 95.

10. A method for manufacturing a solid cosmetic composition, which is a dry manufacturing process, the method comprising:
1) homogeneously mixing porous particles, a powder-type water-soluble active ingredient and a binder;
2) tableting the mixture of 1) to prepare a molded product; and
3) loading an oil-soluble active ingredient into the molded product of 2),
wherein a weight ratio of the binder to the porous particles is 1:5 to 95.
